(19) United States
(12) Patent Application Publication
(10) Pub. No.: US 2010/0266606 A1
(43) Pub. Date: Oct. 21, 2010

(54) ANTIBODY THERAPY FOR HIGHLY PATHOGENIC AVIAN INFLUENZA VIRUS

(75) Inventors: Young Shik Cho, Suwon-si (KR); Gun Woo Ha, Seoul (KR); Jin Sik Oh, Suwon-si (KR); Byung Gi Cho, Suwon-si (KR); Yun Jung Lee, Anyang-si (KR)

Correspondence Address: KNOBBE MARTENS OLSON & BEAR LLP, 2040 MAIN STREET, FOURTEENTH FLOOR, IRVINE, CA, 92614, US

(73) Assignees: STANDARD DIAGNOSTICS, INC., Yongin-si (KR); BIONOTE, INC., Hwaseong-si (KR)

(21) Appl. No.: 12/767,718

(22) Filed: Apr. 26, 2010

Related U.S. Application Data
(63) Continuation of application No. PCT/KR2008/006328, filed on Oct. 27, 2008.

(30) Foreign Application Priority Data
Oct. 26, 2007 (KR) 10-2007-0108519

(51) Int. Cl. A61K 39/42; C07K 16/10; C12N 5/12; C07H 21/04; A61P 31/16

(52) U.S. Cl. 424/147.1; 530/388.3; 530/387.3; 435/346; 536/23.53

(57) ABSTRACT

The present invention relates to a monoclonal antibody against hemagglutinin of highly pathogenic avian influenza virus subtype H5 or functional fragment thereof, a hybridoma producing the monoclonal antibody, and a composition comprising the monoclonal antibody or functional fragment thereof. In addition, the present invention relates to a method for preventing or treating influenza virus infection by administering the composition to a subject, and an assay kit for influenza virus comprising the monoclonal antibody or functional fragment thereof.

### CROSS-REFERENCE TO RELATED PATENT APPLICATIONS

This application is a continuation application under 35 U.S.C. §365(c) of International Application No. PCT/KR2008/006328, filed Oct. 27, 2008 designating the United States. International Application No. PCT/KR2008/006328 was published in English as WO2009/054708 on Apr. 30, 2009. This application further claims the benefit of the earlier filing date under 35 U.S.C. §365(b) of Korean Patent Application No. 10-2007-0108519 filed Oct. 26, 2007. This application incorporates herein by reference the International Application No. PCT/KR2008/006328 including the International Publication No. WO2009/054708 and the Korean Patent Application No. 10-2007-0108519 in their entirety.

### TECHNICAL FIELD

The present invention relates to a monoclonal antibody against hemagglutinin of a highly pathogenic avian influenza virus subtype H5 or a functional fragment thereof, a hybridoma producing the monoclonal antibody, and a composition comprising the monoclonal antibody or functional fragment thereof. In addition, the present invention relates to a method for preventing or treating influenza virus infection by administering the composition to a subject, and an assay kit for influenza virus comprising the monoclonal antibody or functional fragment thereof.

### BACKGROUND

Pathogenic avian influenza virus infects various birds including chicken, turkey, and wild birds, and affects poultry such as chicken and turkey to cause an acute viral infectious disease, of which the spread is fast and pathogenicity is diverse. However, pathogenic avian influenza virus is known not to cause disease symptoms in ducks. Avian influenza virus is classified into the 3 types of high pathogenic, low pathogenic and non-pathogenic avian influenza viruses according to the degree of pathogenicity, among which the high pathogenic avian influenza (HPAI) is classified into “grade A” in the World Organization for Animal Health (OIE) and “a first level domestic animal infectious disease” in Korea. In Korea, an outbreak of highly pathogenic avian influenza (H5N1) has been recently reported (December 2003˜March 2004), and the first outbreak of low pathogenic avian influenza (H9N2) was reported in 1996.

Avian influenza is caused by influenza virus type A that is a member of the family Orthomyxoviridae. Influenza type A is subdivided into serotypes H and N based on two proteins, hemagglutinin (H) and neuraminidase (N). The serotypes of the influenza virus are represented by serotypes H and N (e.g., H9N2), and there are 16H types, each with up to 9 N subtypes, yielding a potential for 144 different H and N combinations. To date, all highly pathogenic strains of avian influenza have been of the H5 and H7 subtypes.

Migratory birds are responsible for the spread of avian influenza, and they have weak or no symptoms even though infected with the virus. However, the low pathogenic avian influenza virus from migratory birds may become highly pathogenic when transmitted to a susceptible poultry population such as chicken or duck. In particular, since chickens are very susceptible to avian influenza virus, the mortality due to dyspnea approaches 100%. Only serotypes H5 and H7 are considered highly pathogenic in poultry.

Human cases of H5N1 influenza, reemerged in Asia in late 2003, were first reported in Hong Kong in 1997 with 6 of 18 human infections resulting in death. By May 2007, there had been 306 reports of human infections with H5N1 virus and 185 deaths. Human symptoms include high fever over 38° C., cough, or other respiratory symptoms such as sore throat and respiratory distress. For diagnostic tests, blood and throat swab specimens are collected, and subjected to rapid antigen test, RT-PCR, Hemagglutination (HA) and hemagglutination-inhibition (HI) tests.

Current strategies to control influenza are still annual immunization with an inactivated whole virus or subunit vaccine.

However, frequent generation of new variants of influenza virus reduces vaccine effectiveness, and the flu vaccines cannot be effective for new influenza viruses infecting humans, such as avian influenza. Thus, there is a need to develop various therapeutic agents for the influenza virus.

There are two types of agents to inhibit the proliferation of the influenza virus. One is amantadine, an M ion channel inhibitor developed by Dupont in 1964, which was used for the treatment of influenza type A virus infection by 1980, but causes adverse effects such as nausea, drowsiness and chronic insomnia (Long J K., Mossad S B., Goldman M P. 2000. Cleve Clin J. med. 67:92-95). Thereafter, the newly developed rimantadine also has a high incidence of adverse effects, although it had a lower potential for causing adverse effects than amantadine (Jefferson et al., 2004. Cochrane Database Syst. Rev. (3): CD001169). The other inhibitor is a neuraminidase inhibitor, zanamivir or oseltamivir, which has been used for the treatment of influenza virus (Dreitlein W B., Maratos J., Brocavich. 2001. Clin Ther. 23:327-355). Zanamivir, currently marketed by GlaxoSmithKline under the trade name Relenza, is an inhalable powder that is inhaled through the nose. Oseltamivir, marketed by Roche under the trade name Tamiflu, is available in an oral preparation, like amantadine. However, Relenza may cause breathing problems to worsen asthma, and Tamiflu may cause adverse effects including nausea and vomiting (McNicholl and McNicholl. 2001. Ann, Pharmacother. 35(1):57-70).

Therefore, the present inventors have made an effort to develop antiviral agents against the influenza virus having less toxicity and fewer adverse effects. They found that highly pathogenic avian influenza virus infection can be treated with monoclonal antibodies against highly pathogenic influenza virus subtype H5.

### SUMMARY

One aspect of the present invention provides a monoclonal antibody against hemagglutinin of highly pathogenic avian influenza virus subtype H5 or functional fragment thereof.

Another aspect of the present invention provides a hybridoma producing the monoclonal antibody.

Still another aspect of the present invention provides a nucleotide encoding the monoclonal antibody or functional fragment thereof.

Still another aspect of the present invention provides an anti-influenza viral composition comprising the monoclonal antibody or functional fragment thereof.

Still another aspect of the present invention provides a method for preventing or treating influenza virus infection by administering to a subject the monoclonal antibody or functional fragment thereof.

Still another aspect of the present invention provides an assay kit for highly pathogenic avian influenza virus subtype H5, comprising the monoclonal antibody or functional fragment thereof.

### DETAILED DESCRIPTION

The present invention relates to a monoclonal antibody against hemagglutinin of highly pathogenic avian influenza virus subtype H5 or functional fragment thereof.

In one embodiment, the monoclonal antibody is a monoclonal antibody produced by a hybridoma KCLRF-BP-00168. In another embodiment, the monoclonal antibody is a monoclonal antibody produced by a hybridoma KCLRF-BP-00169. In still another embodiment, the monoclonal antibody is a monoclonal antibody produced by a hybridoma KCLRF-BP-00170. In still another embodiment, the monoclonal antibody is a monoclonal antibody produced by a hybridoma KCLRF-BP-00171.

As used herein, the term “monoclonal antibody” refers to a protein molecule that is directed by a single antigenic region (single epitope) and specifically binds thereto. Since the monoclonal antibody of an embodiment specifically binds to hemagglutinin of highly pathogenic avian influenza virus subtype H5, the monoclonal antibody is a protein molecule recognizing the hemagglutinin of highly pathogenic avian influenza virus subtype H5.

The major regions of an antibody involved in the recognition of a specific epitope and the formation of antigen-antibody complexes are variable regions of heavy chain and light chain, in particular, the CDR (complementary determining region) attributes to the formation of antigen-antibody complexes. Thus, various aspects of the present invention includes humanized antibodies, chimeric antibodies and deimmunized antibodies of the monoclonal antibody, which comprise variable regions of the monoclonal antibody, especially the CDR.

As used herein, the term “humanized antibody” refers to a polypeptide comprising a modified variable region of a human antibody wherein a portion of the variable region, for example, a portion of the intact human variable domain, has been substituted by the corresponding sequence from a non-human species and wherein the modified variable region is linked to at least another part of another protein, for example, the constant region of a human antibody. As used herein, the term “chimeric antibody” refers to a polypeptide comprising the variable region of a non-human antibody that binds to hemagglutinin linked to at least another part of another protein, for example, the constant region of a human antibody. As used herein, the term “deimmunized antibody” refers to an antibody that is deimmunized by mutation not to activate the immune system of a subject (for example, Nanus et al., J. Urology 170:S84-S89, 2003; WO98/52976; WO00/34317).

In addition, the present invention includes functional fragments of antibody molecules as well as complete forms having two full-length light chains and two full-length heavy chains, as long as they have the above described binding properties. As used herein, the term “functional fragments” refers to antibody molecules that refer to fragments or variable regions retaining at least an antigen-binding capacity, and include V_{H}, V_{L}, scFv, Fab, F(ab′), F(ab′)₂, Fv or the like. The antibody fragments of an embodiment further include multispecific or multivalent structures formed from the above mentioned antibody fragments.

To confirm whether the monoclonal antibody according to some embodiments specifically binds to the hemagglutinin of H5, a hemagglutination inhibition test was performed using an H5 antigen. As a result, all of the four types of monoclonal antibody exhibited hemagglutination inhibition activity against H5N3, and very high neutralizing antibody titers of 2¹⁰ or more in embryonated chicken eggs, indicating that the monoclonal antibodies can neutralize live virus. In addition, when the monoclonal antibody was administered to chicken via intravenous injection, persistence of the antibody was observed for 48 hrs. When administered with the H5N1 virus, the pathogenicity of the H5N1 virus was found to be neutralized.

The present invention also relates to a hybridoma producing monoclonal antibodies.

In accordance with one embodiment, the hybridoma is a hybridoma designated under accession number KCLRF-BP-00168. In accordance with another embodiment, the hybridoma is a hybridoma designated under accession number KCLRF-BP-00169. In accordance with still another embodiment, the hybridoma is a hybridoma designated under accession number KCLRF-BP-00170. In accordance with still another embodiment, the hybridoma is a hybridoma designated under accession number KCLRF-BP-00171.

In some embodiments, a hybridoma is prepared by inactivating avian influenza virus with formalin; immunizing mice with the inactivated avian influenza virus; isolating lymphocytes from the spleen of the mice; and fusing the lymphocytes with myeloma cells. Each of the produced hybridomas was deposited at Korean Cell Line Research Foundation (KCLRF) on Sep. 18, 2007 (accession numbers KCLRF-BP-00168, KCLRF-BP-00169, KCLRF-BP-00170 and KCLRF-BP-00171).

The hybridomas secreting monoclonal antibodies may be cultured in a large scale in vitro or in vivo. In addition, the monoclonal antibodies secreted by the hybridomas may be used without purification, or may be used after being highly purified (e.g., 95% or higher) by methods known in the art in order to obtain the best results. Purification may be carried out using culture fluid or ascites fluid, for example, using gel electrophoresis, dialysis, salting out and chromatography.

In some embodiments of the present invention, for mass production of the monoclonal antibodies, hybridoma cells were intraperitoneally inj ected into mice to be cultured in the peritoneal cavity, and ascites fluid was collected from the mice and purified using a protein A/G gel to isolate the monoclonal antibodies.

In accordance with still another aspect, the present invention relates to a nucleotide encoding the monoclonal antibody.

In accordance with still another aspect, the present invention relates to an anti-influenza viral composition comprising the monoclonal antibody.

The composition prepared by the above method may be administered, for example, injected into a subject to treat or prevent infection with the influenza virus. The composition may be administered in an amount sufficient to prevent or treat infection. The subjects to be administered with the composition of the present invention include all hosts susceptible to infection with influenza virus, for example, humans, birds (chickens, ducks, geese, turkeys, etc.), swine, horses or the like. To prevent and treat infection with the influenza virus in various subjects, the composition according to some embodiments may be prepared as a pharmaceutical composition, feed composition, food composition or the like.

In one embodiment, the anti-influenza viral composition comprising the monoclonal antibody according to some embodiments is provided as a pharmaceutical composition. The pharmaceutical composition may be provided as a therapeutic agent or vaccine. In addition, the composition may include a pharmaceutically acceptable carrier. For oral administration, the pharmaceutically acceptable carrier may include a binder, a lubricant, a disintegrator, an excipient, a solubilizer, a dispersing agent, a stabilizer, a suspending agent, a coloring agent, and a perfume. For injectable preparations, the pharmaceutically acceptable carrier may include a buffering agent, a preserving agent, an analgesic, a solubilizer, an isotonic agent, and a stabilizer. For preparations for topical administration, the pharmaceutically acceptable carrier may include a base, an excipient, a lubricant, and a preserving agent. The pharmaceutical composition may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into a unit dosage form, such as a multidose container or an ampule as a single-dose dosage form.

In another embodiment, the anti-influenza viral composition comprising the monoclonal antibody according to some embodiments is provided as a food (or health functional food) composition. The food composition, in addition to the present microorganism or a culture thereof, may be mixed with suitable carriers and excipients or auxiliary effective ingredients, and may be formulated into powders, granules, tablets, capsules or drinkable form. Suitable carriers, excipients and diluents for use in the formulation of the food composition may include lactose, dextrose, sucrose, sorbitol, mannitol, starch, gum acacia, calcium phosphate, alginate, gum tragacanth, gelatin, calcium silicate, microcrystalline cellulose, polyvinylpyrrolidone, cellulose, water, syrup, methylcellulose, methyl- and propyl-hydroxybenzoate, talc, magnesium stearate and mineral oils. For commercialization, the food composition may further include lubricants, wetting agents, emulsifying agents and suspending agents, antiseptics, sweeteners, or perfumes.

In accordance with still another aspect, the anti-influenza viral composition comprising the monoclonal antibody according to some embodiments may be provided as a feed composition. The feed composition may be prepared in the form of fermented feed, formula feed, pellets, silage or the like

In accordance with still another aspect, the present invention relates to a method for preventing or treating influenza virus infection by administering the composition to a subject suspected of being infected with influenza virus.

As used herein, the term “influenza virus infectious disease” refers to a disease caused by infection with influenza virus, and exemplified by sinusitis, spasmodic asthma, otitis media, cystic fibrosis, bronchitis, pneumonia, and diarrhea (Pitkaranta and Hayden, 1998. Ann. Med.), but is not limited thereto.

As used herein, the term “subject” refers to all animals including human, which had been infected or can be infected with influenza virus. The composition comprising the extract according to some embodiments is administered into a subject to effectively prevent or treat the disease. For example, the composition can treat humans infected with various influenza virus subtypes or variants of human influenza virus. Further, the composition can treat humans infected with various influenza virus subtypes or variants of avian influenza virus. Furthermore, the composition can treat chicken or swine infected with various influenza virus subtypes or variants of avian influenza virus. The composition may be administered in combination with known therapeutic agents for influenza virus infection.

As used herein, the term “prevention” means all of the actions in which influenza virus infection is restrained or retarded by the administration of the composition. As used herein, the term “treatment” means all of the actions in which influenza virus infection has taken a turn for the better or been modified favorably by the administration of the composition.

The composition according to some embodiments may be administered via any of the common routes, as long as it is able to reach a desired tissue. A variety of administration modes are contemplated, including intraperitoneally, intravenously, intramuscularly, subcutaneously, intradermally, orally, intranasally, intrapulmonarily and intrarectally, but the present invention is not limited to these exemplified administration modes. In addition, the pharmaceutical composition may be administered using a certain apparatus capable of transporting the active ingredients into a target cell.

The composition according to some embodiments is administered in a pharmaceutically effective amount. As used herein, the term “pharmaceutically effective amount” refers to an amount sufficient for the treatment of diseases, which is commensurate with a reasonable benefit/risk ratio applicable for medical treatment. An effective dosage of the present composition may be determined depending on the subject and severity of the diseases, age, gender, type of infected virus, drug activity, the patient's drug sensitivity, administration time, administration routes, excretion rates, duration of treatment, simultaneously used drugs, and other factors known in medicine. The composition may be administered as a sole therapeutic agent or in combination with other therapeutic agents, and may be administered sequentially or simultaneously with conventional therapeutic agents. This administration may be provided in single or multiple doses. Taking all factors into consideration, it is important to conduct administration of minimal doses capable of giving the greatest effects with no adverse effects, such doses being readily determined by those skilled in the art.

In still another embodiment, the present invention relates to an assay kit for highly pathogenic avian influenza virus subtype H5, comprising the monoclonal antibodies.

The monoclonal antibodies according to some embodiments may be used for specifically detecting influenza virus through an antigen-antibody complex reaction, as well as removing influenza virus from cells to be infected or infected cells.

In addition to the monoclonal antibodies according to some embodiments, these assay kits may include tools and reagents, which are generally used in the art for immunological analysis. These tools/reagents include, but are not limited to, suitable carriers, labeling substances capable of generating detectable signals, solubilizing agents, detergents, buffering agents, and stabilizing agents. When the labeling substance is an enzyme, the assay kit may include a substrate allowing the measurement of enzyme activity and a reaction terminator. Suitable carriers include, but are not limited to, soluble carriers, for example, physiologically acceptable buffers known in the art, for example, PBS, insoluble carriers, for example polymers such as polystylene, polyethylene, polypropylene, polyesters, polyacrylnitrile, fluorocarbon resin, crosslinked dextran, polysaccharides and magnetic microparticles composed of latex plated with metals, papers, glasses, metals, agarose, and combinations thereof.

Analysis methods for the formation of antigen-antibody complexes include, but are not limited to, immunohistochemical staining, radioimmunoassay (RIA), enzyme linked immunosorbent assay (ELISA), Western blotting, immunoprecipitation assay, immunodiffusion assay, complement fixation assay, FACS, and protein chip assay.

Examples of the detection label that qualitatively or quantitatively determines the formation of antigen-antibody complexes include enzymes, fluorescent substances, ligands, luminescent substances, microparticles, redox molecules and radioactive isotopes, but are not limited thereto. Examples of enzymes available as detection labels include, but are not limited to, β-glucuronidase, β-D-glucosidase, β-D-galactosidase, urase, peroxidase, alkaline phosphatase, acetylcholinesterase, glucose oxidase, hexokinaseandGDPase, RNase, glucose oxidase and luciferase, phosphofructokinase, phosphoenolpyruvate carboxylase, aspartate aminotransferase, phosphenolpyruvatedecarboxylase, and β-latamase Examples of the fluorescent substances include, but are not limited to, fluorescin, isothiocyanate, rhodamine, phycoerythrin, phycocyanin, allophycocyanin, o-phthaldehyde and fluorescamin. Examples of the ligands include, but are not limited to, biotin derivatives. Examples of luminescent substances include, but are not limited to, acridinium esters, luciferin and luciferase. Examples of the microparticles include, but are not limited to, colloidal gold and colored latex. Examples of the redox molecules include, but are not limited to, ferrocene, ruthenium complexes, viologen, quinone, Ti ions, Cs ions, diimide, 1,4-benzoquinone, hydroquinone, K₄W(CN)₈, [Os(bpy)₃]²⁺, [RU(bpY)₃]²⁺, [MO (CN)₈]⁴⁻. Examples of the radioactive isotopes include, but are not limited to, ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Hereinafter, some illustrative Examples are provided for better understanding. However, these Examples are for illustrative purposes only, and the invention is not intended to be limited by these Examples.

### Example 1

### Production of Monoclonal Antibody Against Highly Pathogenic Avian Influenza Virus Subtype H5

Avian influenza antigen was prepared from highly pathogenic avian influenza (A/CK/Korea/ES/03 (H5N1)) in Korea. Avian influenza virus was propagated in 9-day old SPF embryonated chicken eggs for 2 days at 37.6° C., and inactivated with a final formalin concentration of 0.2%, followed by concentration and purification. The inactivated avian influenza virus was centrifuged at 4,000×g for 30 min, and then the supernatant was ultracentrifuged (L8-70M, Beckman, USA) at 70,000×g for 3 hrs. The resulting pellet was resuspended in sucrose (1/25) and subjected to sucrose density gradient ultracentrifugation (100,000×g, 90 min). Fractions showing hemagglutinating activity were isolated and used as an antigen for producing avian influenza monoclonal antibodies.

Balb/c mice were immunized using the antigen that was purified by the above method as an immunogen. Then, spleen cells were isolated and fused with myeloma cells. Hybridomas secreting monoclonal antibodies against avian influenza virus antigen were screened by ELISA, and limiting dilution procedures were performed to establish various monoclonal cell lines. The cell lines were mass-produced, and intraperitoneally injected into Balb/c mice to obtain ascitic fluid containing a large amount of antibodies. IgG antibodies were purified using a protein A/G gel.

Each of the obtained hybridomas was deposited at KCLRF (Korean Cell Line Research Foundation) on Sep. 18, 2007 (Accession Nos. KCLRF-BP-00168, KCLRF-BP-00169, KCLRF-BP-00170 and KCLRF-BP-00171).

### Example 2

### Hemagglutination Inhibition Test

In order to confirm whether the antibodies isolated and purified in Example 1 specifically reacted with hemagglutinin of H5, a Hemagglutination Inhibition Test was performed using H5 antigen. The Hemagglutination Inhibition Test was performed in accordance with the procedures of the World Organization for Animal Health (OIE). Briefly, the purified monoclonal antibodies were serially diluted two-fold with PBS (pH7.2), and added to the wells of a V bottom microplate (25 μl per well), and H5N3 virus of 4HAU (25 μl) was added to each well and neutralized for 30 min. Then, 25 μl of 1% chicken red blood cells were added thereto, and the monoclonal antibody titers were expressed as the reciprocal of the highest dilution that inhibited hemagglutination of erythrocytes.

As a result, four types of monoclonal antibodies were found to show hemagglutination inhibition activity against H5N3 (Table 1).

### Example 3

### Neutralization Test in Embryonated Chicken Eggs

In order to confirm whether four types of the monoclonal antibodies according to some embodiments of the present invention neutralized live virus, a neutralization test was performed in embryonated chicken eggs.

Each antibody (singly or mixed with each other) was serially diluted two-fold with PBS, and mixed with 2000EID₅₀/0.1 ml of H5N3 virus at an equivalent amount, followed by incubation at 37□ for 1 hr for neutralization. 9 day-old embryonated chicken eggs were inoculated with the antibodies, and cultured for 4 days. After completing the culture, allantoic fluid of the embryonated chicken egg was harvested, and the presence of virus was examined. In order to confirm the presence of virus, 50 μl of allantoic fluid and 50 μl of 5% chicken red blood cells were mixed with each other to examine hemagglutination. The neutralizing antibody titers were calculated as the reciprocal of the highest dilution that completelyneutralized the virus. As a result, all of the hemagglutinin-specific antibodies showed high neutralizing antibody titers of 2¹⁰ or higher, indicating that they can neutralize live virus (Table 1). The mixed antibodies also showed very high antibody titer.

TABLE 1
Hemagglutination inhibition titer and neutralizing
antibody titer of monoclonal antibody

| Clone No. | | HI | Nucleoprotein | SN |
|---|---|---|---|---|
| (Conc.) | | (H5N3 antigen) | ELISA | (H5N3 virus) |
| 25-1 | (1 mg/ml) | 2(9) | Negative | >2(10) |
| 51 | (1 mg/ml) | 2(10) | Negative | >2(10) |
| 1290 | (1 mg/ml) | 2(9) | Negative | 2(10) |
| 12E7-3B9 | (1 mg/ml) | 2(10) | Negative | 2(10) |

| | | | |
|---|---|---|---|
| (25-1) + (51) | Not tested | Not tested | >2(10) |
| (25-1) + (16) | Not tested | Not tested | >2(10) |
| (25-1) + (B23) | Not tested | Not tested | >2(10) |
| (25-1) + (1290) | Not tested | Not tested | >2(10) |
| (51) + (16) | Not tested | Not tested | >2(10) |
| (51) + (B23) | Not tested | Not tested | >2(10) |
| (51) + (1290) | Not tested | Not tested | >2(10) |
| (16) + (B23) | Not tested | Not tested | 2(8) |
| (16) + (1290) | Not tested | Not tested | >2(10) |
| (B23) + (1290) | Not tested | Not tested | >2(10) |

### Example 4

### Antibody Persistence in Chicken

An antibody persistence test was performed in order to confirm whether the monoclonal antibodies according to some embodiments of the present invention have the long-term antibody persistence to improve therapeutic effects in chicken.

7 week-old SPF chickens were inoculated with 1 ml of H5-speicific 1290 antibody (1 mg/ml) (2⁹HI titer) by intravenous and intramuscular injection. Then, the hemagglutination inhibition test against H5N3 was performed at 3, 24, and 48 hrs after inoculation, so as to confirm whether the antibody titers maintained.

In the intravenous injection group, the passively administered antibody remained for 48 hrs (Table 2)

TABLE 2
Antibody persistence test

| | Hemagglutination inhibition titer | | | | |
|---|---|---|---|---|---|
| | Before | After 3 | After 24 | After 48 | After 72 |
| Injection route | inoculation | hrs | hrs | hrs | hrs |
| Intravenous | 2-fold or | 2⁴ | 2³ | 2¹ | 2-fold or |
| injection, | less | | | | less |
| 1 mg/ml, 2⁹ HI | | | | | |
| titer | | | | | |
| Intramuscular | 2-fold or | 2-fold | 2-fold | 2-fold | 2-fold or |
| injection, | less | or less | or less | or less | less |
| 1 mg/ml, 2⁹ HI | | | | | |
| titer | | | | | |

### Example 5

### Therapeutic Effects in Chicken

An antibody persistence test was performed in order to confirm whether the monoclonal antibodies according to some embodiments of the present invention have the long-term antibody persistence to improve therapeutic effects in chicken, when 4 types of the monoclonal antibodies were mixed with each other in an equivalent amount and injected to 7-week old SPF chickens. Briefly, H5N1 virus (ES/03 week) and the selected 4 types of the monoclonal antibodies were mixed with each other in an equivalent amount, and immediately injected to chickens. Then, the onset of H5 symptoms was examined to confirm the therapeutic or prophylactic effects in vivo.

As a result, in the group inoculated with both H5N1 virus and H5 monoclonal antibody, pathogenicity was neutralized, and thus 3 of 8 chickens died. In the group inoculated with H5N1 virus only, 6 of 6 chickens died on day 2. Thus, it can be seen that the monoclonal antibodies showed neutralizing activity (Table 3).

TABLE 3
Neutralization test in chicken

| | Mixture | | | | | | | | | | | | |
|---|---|---|---|---|---|---|---|---|---|---|---|---|---|
| | | H5 | | | | | | | | | | | |
| | H5N1 | monoclonal | Injection | Inoculation | Death | | | | | | | | |
| Section | (Es/03) | Ab | route | No. | DPI 1 | DPI 2 | DPI 3 | DPI 4 | DPI 5 | DPI 6 | DPI 7 | DPI 8 | Total |
| Neutralizing | 0.1 ml | 0.1 ml | Intravenous, | 8 | 0 | 0 | 0 | 1 | 0 | 2 | 0 | 0 | 3 |
| group | (2000 | | 0.2 ml/head | heads | | | | | | | | | deaths |
| | ELD50/ | | | | | | | | | | | | |
| | 0.1 ml) | | | | | | | | | | | | |
| Non-Neutralizing | 0.1 ml | 0.1 ml | Intravenous, | 6 | 0 | 6 | — | — | — | — | — | — | 6 |
| group | (PBS) | | 0.2 ml/head | heads | | | | | | | | | deaths |

The monoclonal antibodies against highly pathogenic avian influenza virus subtype H5 according to some embodiments of the present invention can be used for the treatment of highly pathogenic avian influenza virus subtype H5, while having less toxicity and fewer adverse effects.

What is claimed is:

1. A monoclonal antibody capable of acting against hemagglutinin derived from avian influenza virus subtype H5, wherein the monoclonal antibody is produced by a hybridoma selected from the group consisting of accession Nos. KCLRF-BP-00168, KCLRF-BP-00169, KCLRF-BP-00170 and KCLRF-BP-00171.

2. The monoclonal antibody according to claim 1, wherein the monoclonal antibody comprises one or more selected from the group consisting of a humanized antibody, a chimeric antibody, and a deimmunized antibody.

3. The monoclonal antibody according to claim 1, wherein the antibody comprises a fragment of an intact antibody produced by the hybridoma.

4. The monoclonal antibody according to claim 3, wherein the fragment comprises one or more selected from the group consisting of V_{H}, V_{L}, scFv, Fab, F(ab′), F(ab′)₂ and Fv.

5. A hybridoma selected from the group consisting of accession Nos. KCLRF-BP-00168, KCLRF-BP-00169, KCLRF-BP-00170 and KCLRF-BP-00171.

6. A nucleotide encoding the monoclonal antibody of claim 1.

7. The nucleotide according to claim 6, wherein the monoclonal antibody comprises one or more selected from the group consisting of a humanized antibody, a chimeric antibody, and a deimmunized antibody.

8. The nucleotide according to claim 6, wherein the antibody comprises a fragment of an intact antibody produced by the hybridoma.

9. The nucleotide according to claim 8, wherein the fragment comprises one or more selected from the group consisting of V_{H}, V_{L}, scFv, Fab, F(ab′), F(ab′)₂ and Fv.

10. An anti-influenza viral composition comprising the monoclonal antibody of claim 1.

11. The anti-influenza viral composition according to claim 10, wherein the monoclonal antibody comprises one or more selected from the group consisting of a humanized antibody, a chimeric antibody, and a deimmunized antibody.

12. The anti-influenza viral composition according to claim 10, wherein the antibody comprises a fragment of an intact antibody produced by the hybridoma.

13. The anti-influenza viral composition according to claim 12, wherein the fragment comprises one or more selected from the group consisting of V_{H}, V_{L}, scFv, Fab, F(ab′), F(ab′)₂ and Fv.

14. The anti-influenza viral composition according to claim 10, wherein the composition further comprises a pharmaceutically acceptable carrier.

15. A method of preventing or treating influenza virus infection, the method comprising:
administering the composition of claim 10 in an amount sufficient to prevent or treat infection to a subject in need of such prevention or treatment.

16. An assay kit for detecting avian influenza virus subtype H5, the kit comprising the monoclonal antibody of claim 1.

17. The assay kit according to claim 16, wherein the monoclonal antibody comprises one or more selected from the group consisting of a humanized antibody, a chimeric antibody, and a deimmunized antibody.

18. The assay kit according to claim 16, wherein the antibody comprises a fragment of an intact antibody produced by the hybridoma.

19. The assay kit according to claim 18, wherein the fragment comprises one or more selected from the group consisting of V_{H}, V_{L}, scFv, Fab, F(ab′), F(ab′)₂ and Fv.
